(19) United States
(12) Patent Application Publication
(10) Pub. No.: US 2023/0338241 A1
(43) Pub. Date: Oct. 26, 2023

(54) PROCESS OF OBTAINING TEMPORARY REFLECTIVE TATTOO AND TEMPORARY REFLECTIVE TATTOO OBTAINED

(71) Applicant: PRISMALITE IMP. EXP. E INDÚSTRIA DE FILMES ÓPTICOS LTDA, Sao Paulo (BR)

(72) Inventor: Jayme Alípio de Barros Filho, Sao Paulo (BR)

(21) Appl. No.: 18/127,765

(22) Filed: Mar. 29, 2023

(30) Foreign Application Priority Data
Apr. 26, 2022 (BR) 10 2022 007953 6

(51) Int. Cl. A61K 8/02; B32B 29/00; B32B 7/12; B41F 15/14; A61Q 1/02; A61K 8/26; A61K 8/25

(52) U.S. Cl. CPC A61K 8/0245; B32B 29/002; B32B 7/12; B41F 15/14; A61Q 1/025; A61K 8/26; A61K 8/25; B32B 2255/28; B32B 2255/12; A61K 2800/72; A61K 2800/43; A61K 2800/87

(57) ABSTRACT

A process (10) for obtaining a temporary reflective tattoo of the transferable type for the skin of users (US) by means of a transfer process through the application of humidity on the support paper. Said process for obtaining the reflective temporary tattoo (10) is subdivided into two phases: phase (F1) application of a reflective fluidizing solution (12) on the water transferable substrate (11) for serigraphic use, and phase (F2) application of hypoallergenic glue (14) on the printed water transferable substrate (11). The temporary tattoo (10A) with reflective image (IG) is provided with a reflectivity index of at least 100 CPL.

### CROSS REFERENCE TO RELATED APPLICATION

This application claims the benefit of and takes priority from Brazilian Patent Application No. BR 10 2022 007953 6 filed on Apr. 26, 2022, the contents of which are herein incorporated by reference.

### TECHNICAL FIELD OF THE INVENTION

The present invention patent deals with a process for obtaining a temporary reflective tattoo and a temporary reflective tattoo obtained of the transferable type to the user's skin and whose predictability of aluminized reflective pigments allows obtaining a temporary tattoo differentiated from the market composing an individual artistic language that enables the temporary use of reflective ornaments on the skin that, in turn, can be instruments of advertising media, social ornament and even a means of signaling motorcyclists, cyclists and the like.

### BACKGROUND OF THE INVENTION

It is known that nowadays temporary tattoos are growing since the definitive tattoo practice is invasive by the application of ink in the dermis region injected through fine needles that penetrate around 2 mm of skin until it reaches the adequate place.

The main advantage of temporary tattoos consists in the fact that they are easily removable, thus enabling a quick and practical replacement, besides allowing the practice of testing a more suitable place to receive the desired ornament, and within the options of temporary tattoos it is foreseen in the market the tattoo being performed by applying paste derived from henna extract which consists of a substance extracted from a plant with a red-orange coloration and durability of up to 30 days depending on the skin type. The drawings are applied on the user's skin by an artist and are limited by shapes and colors.

Another form of temporary tattoo is the type applied by airbrush, using anti-allergic ink and whose durability of the ornament on the skin can vary from 5 to 7 days, being widely used in children's events.

The temporary tattoo applied by transfer process through decal comprises a copy of an image printed by a printer on an adhesive paper previously glued on a certain support paper and through the application of water the image transfer is performed on the user's skin.

The durability is reduced and depends on the user washing the ornamented place.

In general, these temporary transfer tattoos have black or colored pigments, but are opaque, and are therefore ornaments with characteristics found in many specimens.

### PRIOR ART

In research performed in specialized databases, documents were found referring to temporary tattoos as presented in document no. PI0603285-0 that deals with a self-adhesive, temporary and removable tattoo that stands out for being composed of a layer of hypoallergenic transparent adhesive previously cut according to the design reproduced, and optionally has a base that forms a layer with cut paper to give contour to the design; the tattoo, either directly on the hypoallergenic transparent adhesive or using associated with it a base with a layer of cut paper, is coated, according to the predetermined outlines or designs, with glitter-based materials (colored), metals (colored) and/or plastics (transparent and/or colored), crystals (colored) and semi-precious stones (colored), particularly applied by gluing, not discarding the option of using precious stones, and the referred materials combined form drawings with different motives, obeying the predetermined drawing, application done by gluing on the dermis, nails or hair, through manual method; the tattoo is supplied preferably applied on cardboard paper.

Document No. US2010313774 discloses a waterless temporary tattoo produced from a film having a pre-treated printing surface and an opposing adhesive surface. An opaque tattoo image is printed on the film printing surface using a screen-printing process and then dried or cured using forced heat or air drying. The completed screen-printed tattoo image is then rotary cut to conform the film to the shape of the tattoo image to produce the completed waterless temporary tattoo.

Document No. US2005073644A1 discloses a temporary under-eye anti-reflective sports tattoo composed of a solid dermatological composition with anti-reflective properties.

The composition forms a film that is deposited on a person's skin under the eyes and on the cheekbones by a dry transfer method. The film thus applied reduces eye glare and increases contrast sensitivity. By incorporating team or company logos, trademarks or the like into the film, the present invention also serves as an advertising mechanism for companies or a mechanism to promote team spirit. The film can hold a predefined shape, has adhesive properties, is water resistant, has fast coloring, and is heat resistant. The composition comprises oil, a gelling compound, colorants, and a mixture of wax and carbon.

Thus, it is a fact that the documents cited in the paragraphs above, despite belonging to the same field of application, do not have any of the characteristics of the object now being improved, thus ensuring that it meets the legal requirements for patentability.

### OBJECTIVES OF THE INVENTION

It is an objective of the invention to present a process for obtaining a temporary reflective tattoo and temporary reflective tattoo obtained of the transferable type to the skin of the user and whose predictability of aluminized reflective pigments differentiate from the temporary tattoos available in the market.

It is an objective of the invention to present a temporary reflective tattoo that is transferable to the skin of the user and that can be used as a means of signaling motorcyclists, cyclists and the like, since the light reflection obtained predicts a reflectivity index of at least 100 CPL, which is appropriate for said circumstances.

It is an objective of the invention to present a temporary reflective tattoo transferable to the skin of the user used as an advertising element in diverse events such as exhibitions, car shows and others.

It is an objective of the invention to present a temporary reflective tattoo transferable to the skin of the user used as an ornamental element, being a new form of individual artistic language.

### DESCRIPTION OF FIGURES

To complement the present description to obtain a better understanding of the characteristics of the present invention and in accordance with a preferred practical realization thereof, the description is accompanied, in annex, by a set of drawings, where, in an exemplified, although not limitative, manner, it has been represented:

FIG. 1 shows a schematic view of the steps that make up the process of obtaining a reflective temporary tattoo; and

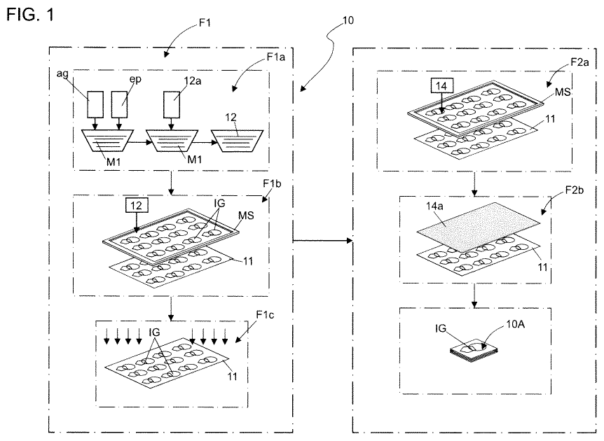

FIG. 2 reveals the temporary reflective tattoo obtained and transferred to the user's skin.

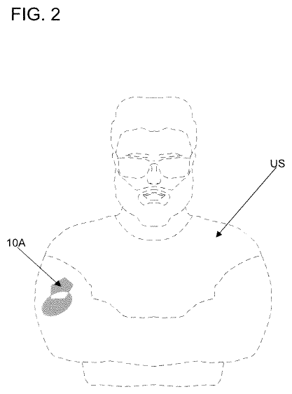

### DESCRIPTION OF THE INVENTION

The present invention patent relates to the “PROCESS FOR OBTAINING REFLECTIVE TEMPORARY TATTOO AND REFLECTIVE TEMPORARY TATTOO OBTAINED”, of the transferable type to the skin of users (US) by means of a transfer process through application of moisture on the backing paper.

According to the present invention, the process of obtaining temporary reflective tattoo (10) is subdivided into two phases, being phase (F1) application of reflective fluidizing solution (12) on the hydro transferable substrate (11) for screen printing use and phase (F2) application of hypoallergenic glue (14) on the printed hydro transferable substrate (11), with each phase (F1) and (F2) presenting a set of steps, which are:

Phase (F1) application of reflective fluidizing solution (12) to the hydro transferable substrate (11) for screen printing use provides for the following steps:
Step (F1a)—The formation of the reflective fluidizing solution (12) is started by mixing: (i) 100 ml of water (ag) and (ii) 7 grams of film forming thickener (ep) forming a mixture (M1) receiving 70 grams of aluminized reflective pigments (12a), preferably in the size of 30˜50 microns, 100% optical glass with 1.93 refractive index and 95% of esterification degree mixed manually for 10 minutes, forming a paste of reflective fluidizing solution (12) with a viscosity between 10,000 to 13,000 Cps needed for the screen printing process;
Step (F1b)—This paste (12) is spread with the help of a serigraphic squeegee over the serigraphic matrix (MS) for the image transfer (IG) to the hydro transferable substrate (11), preferably gummed paper;
Stage (F1c)—The paper (11) follows for curing with the aid of ‘UV’ curing lamps, and the thickener also helps in drying the mixture and forms a film on the paper (11) and on the reflective pigment (12a);

Step (F2) of applying hypoallergenic glue (14) on the hydro transferable substrate (11) printout provides for the following steps:
Step (F2a)—A layer of hypoallergenic glue (14) supplied in the same viscosity as screen printing inks is applied through the same screen-printing screen (MS) for juxtaposition on the ink applied to the paper (11);
Step (F2b)—After applying the glue (14) the print receives a protective film (14a) to protect the glue, finalizing the card by obtaining the temporary tattoo film (10A) with reflective image (IG) provided with a reflectivity index of at least 100 CPL;
Step (F2c)—After the commercial cards are cut with a guillotine, the temporary tattoos (10A) with reflective image (GI) are sent to packaging.

To apply the temporary reflective tattoo film (10A) on the skin of the user (US), the protective film (14a) is removed and the gummed paper (11) is applied to the desired location by applying a wet sponge on the paper (11) for 20 seconds, following with the removal of the paper (11) until the reflective image (IG) fixes on the skin for 5 to 7 days, and the removal of the temporary reflective tattoo (10A) can be effected at any time by simply applying moisturizing oil.

It is certain that when the present invention is put into practice, modifications may be introduced regarding certain details, without deviating from the fundamental principles that are clearly substantiated in the claim, it being understood that the terminology used was not intended as a limitation.

What is claimed is:

1. A process for obtaining temporary reflective tattoo, of the type transferable to the user's skin (US) by means of a transfer process through the application of humidity on the support paper; characterized by the process of obtaining the temporary reflective tattoo (10) being subdivided into two phases, being phase (F1) application of reflective fluidizing solution (12) on the hydro transferable substrate (11) for serigraphic use and phase (F2) application of hypoallergenic glue (14) on the printed hydro transferable substrate (11); phase (F1) application of reflective fluidizing solution (12) on the hydro transferable substrate (11) for silkscreen use provides for step (F1a) with the formation of the reflective fluidizing solution (12) by mixing (i) 100 ml of water (ag) and (ii) 7 grams of film forming thickener (ep) configuring a mixture (M1) receiving 70 grams of aluminized reflective pigments (12a) in the size of 30 ˜50 microns, 100% optical glass with 1.93 refractive index and 95% of esterification degree mixed manually for 10 minutes, forming a paste of reflective fluidizing solution (12) with viscosity between 10,000 to 13. 000 Cps, necessary for the serigraphic process; step (F1b) where the paste (12) is spread with the help of a serigraphic squeegee over the serigraphic matrix (MS) for the transfer of the image (IG) to the hydro transferable substrate (11), preferably gummed paper; step (F1c) in which the paper (11) is cured with the help of UV curing lamps, and the thickener also helps in drying the mixture and forms a film on the paper (11) and on the reflective pigment (12a); The phase (F2) of application of hypoallergenic glue (14) on the printed hydro transferable substrate (11) presents step (F2a) where a layer of hypoallergenic glue (14) provided in the same viscosity of screen printing inks is applied through the same screen printing screen (MS) for the juxtaposition on the ink applied on the paper (11); step (F2b) after applying the glue (14) the pattern receives a protective film (14a) to protect the glue, finalizing the card by obtaining the temporary tattoo film (10A) with reflective image (IG); step (F2c) after cutting the commercial cards with a guillotine, the temporary tattoos (10A) with reflective image (IG) go to packaging.

2. The temporary reflective tattoo obtained according to claim 1, characterized by reflective image (IG) of the obtained temporary tattoo (10A) having a reflectivity index of at least 100 CPL.
